(19) United States
(12) Patent Application Publication
(10) Pub. No.: US 2003/0065268 A1
(43) Pub. Date: Apr. 3, 2003

(54) Optical computed tomography in a turbid media

(75) Inventors: Kun Chen, Charlotte, NC (US); Lev T. Perelman, Brookline, MA (US); Qingguo Zhang, Malden, MA (US); Ramachandra R. Dasari, Lexington, MA (US); Michael S. Feld, Waban, MA (US)

Correspondence Address: BOWDITCH & DEWEY, LLP, 161 WORCESTER ROAD, P.O. BOX 9320, FRAMINGHAM, MA, 01701-9320, US

(73) Assignee: Massachusetts Institute of Technology, Cambridge, MA

(21) Appl. No.: 09/848,767

(22) Filed: May 4, 2001

Related U.S. Application Data
(60) Provisional application No. 60/201,938, filed on May 5, 2000.

(51) Int. Cl. A61B 6/00

(52) U.S. Cl. 600/473

(57) ABSTRACT

Photon migration methods are employed to image absorbing objects embedded in a turbid medium such as tissue. For improved resolution, early arriving photons are detected to provide data with image reconstruction based on optical computed tomography (CT). The CT method is generalized to take into account the distributions of photon paths. A point spread function (PSF) is expressed in terms of the Green's function for the transport equation. This PSF provides weighting functions for use in a generalized series expansion method. Measurements of turbid medium with scattering and absorption properties included coaxial transmission scans collected in two projections. Blurring associated with multiple scattering was removed and high-resolution images can be obtained.

### RELATED APPLICATIONS

This application claims the benefit of U.S. patent application Ser. No. 60/201,938 filed May 5, 2000. The entire teachings of the above application is incorporated herein by reference.

### GOVERNMENT SUPPORT

The invention was supported, in whole or in part, by Grant No. P41-RR02594 from the National Institutes for Health. The Government has certain rights in the invention.

### BACKGROUND OF THE INVENTION

X-ray computed tomography (CT) has been very successful in imaging internal structures of the human body. It has provided an accurate, micro-resolution and real-time medical imaging tool for clinical use. However, the use of X-rays has several disadvantages. The contrast is low for certain kinds of tumors, such as early stage breast cancer. The misdiagnose rate for X-ray mammography is high and X-rays are mutagenic. In recent years, imaging techniques based on optical photons have attracted significant interest. In the spectral region between 700 and 900 nm, called the therapeutic window, photons do not give rise to mutagenic effects, and they can penetrate deeply into tissues, due to the weak absorption of light at these wavelengths. Sensitivity to optical contrast is high and spectroscopic information can be obtained. Further, contrast can be enhanced by injecting exogenous dyes which target tumor cells. Thus, the information provided by optical photons can complement that of X-ray CT, and perhaps provide an alternative diagnostic tool for detecting tumors and other abnormalities inside the body.

### SUMMARY OF THE INVENTION

The major obstacle to optical imaging is the high turbidity of human tissues. Photons injected into tissue undergo multiple scattering events before they are detected. To extract spatial information, one has to disentangle the effects of scattering. Various optical imaging approaches have been developed to deal with this problem. Each of these approaches has advantages and disadvantages. Imaging techniques utilizing ballistic photons, which are very good for imaging up to one millimeter inside the tissue, do not work in the case of deep tissue imaging. Several image reconstruction schemes have been devised to deconvolve turbidity and improve spatial resolution. Finite element methods and finite difference methods have been developed in the time domain and the frequency domain, respectively. Filtered backprojection has been applied to CW imaging. Weighting factors for the contribution of individual voxels to the measurement, first proposed in X-ray CT, have also been calculated using Monte Carlo simulations and employed in the inverse model. The present invention relates to the use of series expansion methods to the optical regime. Using an early time detection approach, three dimensional images of a tissue can be reconstructed by taking into account the effects of turbidity.

The problem can be understood by comparing the propagation of optical photons and X-rays in human tissue. As it is well known, X-rays traversing the body (FIG. 1a) travel along straight lines. In contrast, due to scattering, the propagation of optical photons has a three dimensional spread. The distribution of optical photon paths can be visualized as a tube connecting the source and the detector (FIG. 1b). The width of the cross section of the tube varies according to the time at which arriving photons are collected.

Several features of the photon path distribution can be noted:

First, in the time-of-flight limit this tube reduces to a straight line, and the problem is reduced to that of conventional X-ray CT. However the signal produced by these so-called ballistic photons is extremely weak, and essentially non-detectable for thick tissues; Second, for early detection times (a few hundred ps after the time of flight), the tube is still very narrow and the photon paths are well defined. The transmitted signals are significant, and spatial information is still highly preserved; and Third, at late detection times (several ns), the tube can completely fill the organ of interest, and spatial resolution is significantly reduced. This regime is referred to as the diffusive limit.

By replacing the straight line paths of X-rays with the narrow tubes of the early time photon path distribution, an optical CT procedure can be employed that is a modification of that used in X-ray CT.

In the optical regime, the early arriving photons are analogous to X-ray photons. They undergo a smaller number of scattering events in comparison with highly diffusive photons, and thus preserve a significant amount of spatial information. Yet signal levels for early arriving photons can be relatively high. Measurements taken using early time detection have higher resolution compared to those obtained with continuous wave (CW) and frequency-domain techniques. Sharp images can be reconstructed using the concept of photon path density. As is well known, the diffusion approximation solution does not well describe the early arriving photons. The predictions of the diffusion approximation with a point spread function up to the t−t₀≈600 ps time window are inadequate for correct image reconstruction. A point spread function (PSF) that takes into account causality produces much better results. The correct form of the point spread function for early time plays an essential role for image reconstruction.

The image reconstruction method of the present invention is based on the use of a series expansion method in the optical regime, where scattering is dominant and the distribution of photon paths between source and detector must be taken into account. To accomplish this, a PSF is used to generate a weighting function matrix. Previously, weighting functions have been discussed and calculated using the diffusion approximation, the microscopic Beer-Lambert law, and Monte Carlo simulations. However, the use of the PSF provides guidance into the choice of weighting functions. The physical interpretation is clearer in terms of the PSF, and different theories about photon migration can be tested because they predict different PSF's. Furthermore, as shown in Eq. (12) below, there are actually two sets of weighting functions, one for scattering contrast and one for absorption contrast. For example, tumors in breast tissue exhibit both absorption and scattering contrast. The early portion of the photon migration curve is more sensitive to the scattering contrast than the absorption contrast.

The resolution of optical CT is restricted by several factors, such as the effects of scattering and the underdetermined nature of the reconstruction procedures. Additionally, the total number of projections and measurements can be increased, and a fan-beam geometry can be used to improve data collection efficiency. Fiberoptic systems can be used for delivery and/or collection of light from the tissue of a patient under examination. Refined time-domain photon migration instruments, implemented with a computer using reconstruction programs can provide optical CT images with high quality in the breast, the brain and elsewhere in the body.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and 1B are schematic diagrams employing an algebraic reconstruction technique for Optical CT in which the sample under study is divided into N×N×N vowels and the absorption distribution is represented by the average absorption within each voxel.

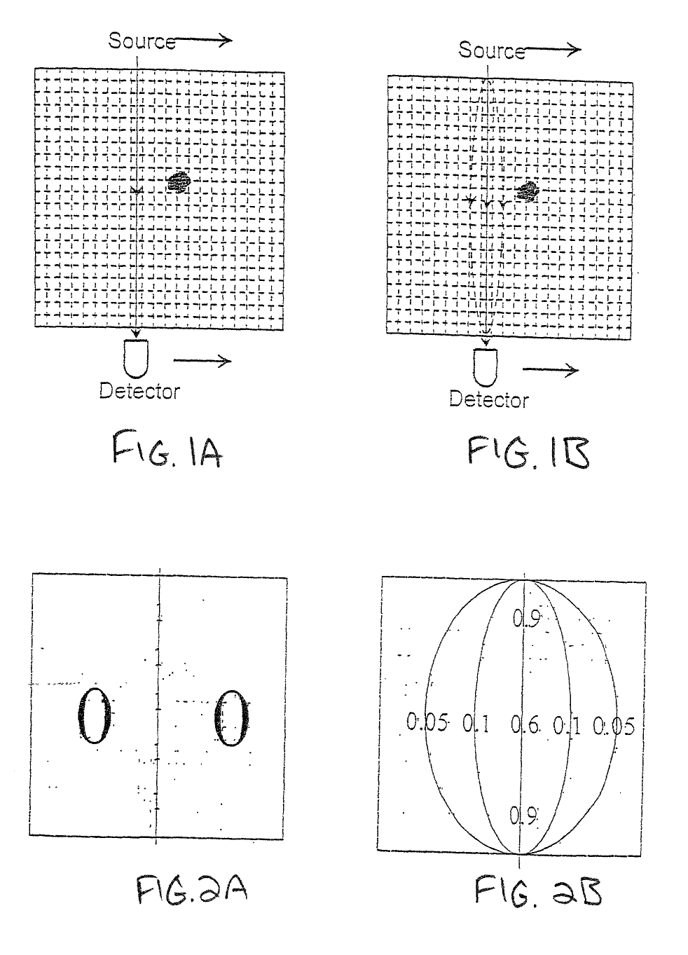

FIGS. 2A and 2B illustrate each voxel being assigned a weighting factor including for the X-ray, in which the voxel on the trajectory has 100% contribution, while off the trajectory has 0% contribution and for the optical photons, the weighting factor is determined by the photon path distribution, respectively.

FIGS. 3A and 3B are schematic diagrams of systems for performing optical computed tomography in accordance with the invention.

FIG. 4 illustrates an example of the dimension of the scattering medium and the scanning geometry.

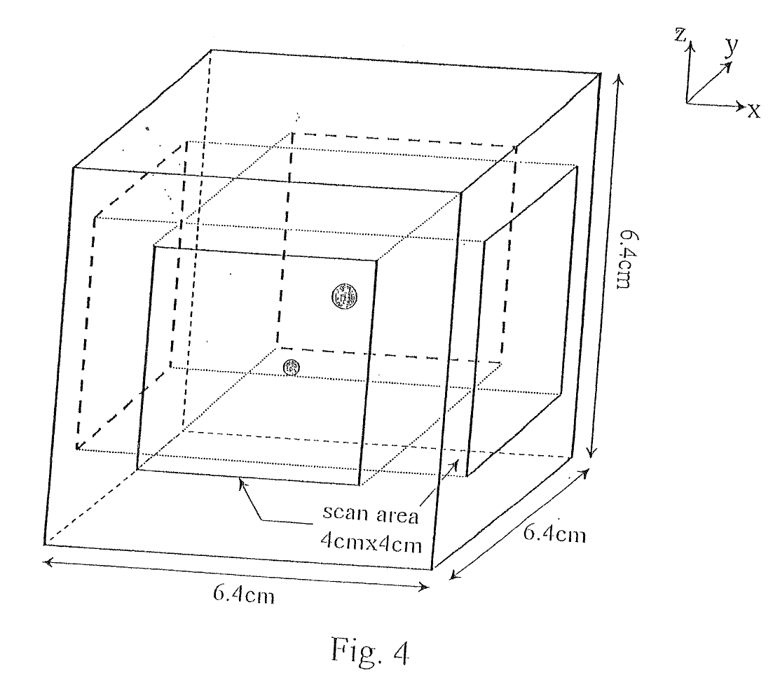

FIGS. 5A and 5D are contour maps of the intensities for 1-object (a)-(b) and 2-objects (c)-(d) with time windows of: (a)-(b) Δt 534 ps, width 50 ps; (c)-(d) Δt =607 ps, width 50 ps.

FIGS. 6A-6D are point spread functions in which the source detector are at (0.-3.2.0) cm, and (0.3.2.0) cm, respectively, and four combinations are given, (A) the side view of the z=0 plane, calculated with the diffusion approximation, (B) the side view of the Z=0 plane, calculated with the causality correction: (C) the top view of the y=0 plane, calculated with the diffusion approximation; (D) the top view of the y=0 plane, calculated with the causality correction.

FIGS. 7A-7D are reconstructed images with one embedded object including (A) reconstruction with direct X-ray algorithm; (B) reconstruction with diffusion approximation; (C) reconstruction with causality correction; (D) the exact configuration.

FIGS. 8A-8D are images of two embedded objects including (A) Reconstruction with direct X-ray algorithm; (B) reconstruction with diffusion approximation; (C) reconstruction with causality correction; (D) the exact configuration. Reconstruction with the direct X-ray algorithm does not resolve the two embedded objects. On the other hand, the reconstructions with diffusion approximation overdo the de-convolution and result in smaller images. The smaller object is invisible from the 3D view.

FIGS. 9A-9D show the contour map diagram of four slices of the reconstructed image in FIG. 8C where the slices are (a) Z=10 mm; (b) Z=−6 mm; (c) Z=2 mm; and (d) Z=10 mm.

FIG. 10 illustrates a process sequence of a preferred embodiment of the invention.

### DETAILED DESCRIPTION OF THE INVENTION

The representation of the attenuation of the X-ray intensity through the human body is well established. Assume the human tissue under study has an attenuation distribution μ_{α}^{X}(r′) for a monochromatic X-ray. Then the transmitted intensity of the X-ray beam across the body is:

$$I ( r ) = {I}_{0} exp [ - {\int }_{{r}_{0}}^{r} ⅆ l {μ}_{a}^{x} ( {r}^{′} ) ] . \qquad ( 1 )$$

where the integral is along the straight line connecting the source at r₀ and the detector at r. Equation (1) can be rewritten as:

$$- [ l n l ( r ) - I n {I}_{0} ] = {\int }_{{r}_{0}}^{r} ⅆ l {μ}_{a}^{x} ( {r}^{′} ) . \qquad ( 2 )$$

The above line integral is often referred to as the Radon transform.

Unlike X-rays, near infrared light in human tissue undergoes strong scattering and does not follow straight-line paths. Optical photons undergo multiple scattering events before they are detected or absorbed by the tissue. The distribution of photon paths in a uniform scattering medium has been studied using various approaches. It has been established that the distribution of the photon paths is narrow at early detection times, but spreads out as time increases.

Generally, the presence of tumors creates optical heterogeneity which appears as a local variation of the scattering and the absorption properties of the tissue. For early stage tumors, these changes can be considered as small perturbations.

The propagation of near infrared photons in human tissue is well described by the transport equation. For a medium with scattering distribution μₛ(r), and absorption distribution μ_{α}(r), the radiance L(r,ŝ,t) satisfies:

$$\frac{1}{c} \frac{\partial L ( r , \hat{s} , t )}{\partial t} + \hat{s} \cdot ∇ L ( r , \hat{s} , t ) = - [ {μ}_{a} ( r ) + {μ}_{s} ( r ) ] L ( r , \hat{s} , t ) + {μ}_{s} ( r ) {\int }_{4} {\int }_{π} L ( r , \hat{s} , t ) {f}_{r} ( \hat{s} \cdot {\hat{s}}^{′} ) ⅆ {Ω}^{′} + Q ( r , \hat{s} , t ) \qquad ( 3 )$$

and the normalized differential scattering cross-section ƒᵣ(ŝ·ŝ′), often referred to as the phase function, satisfies:

$${\int }_{4} {\int }_{π} {f}_{r} ( \hat{s} \cdot {\hat{s}}^{′} ) ⅆ {Ω}^{′} = 1 \qquad ( 4 )$$

In Eq. (3), c is the speed of light in the medium, and Q(r,ŝ,t) is the source term. For a perturbative system, the distribution of absorption, scattering and phase function have small variations of the form:

$$\{ \begin{matrix}{μ}_{a} ( r ) = {μ}_{a} + {δμ}_{a} ( r ) , \\ {μ}_{s} ( r ) = {μ}_{s} + {δμ}_{s} ( r ) , \\ {μ}_{s} ( r ) {f}_{r} ( \hat{s} \cdot {\hat{s}}^{′} ) = {μ}_{s} f ( \hat{s} \cdot {\hat{s}}^{′} ) + δ {\tilde{μ}}_{s} ( r , \hat{s} \cdot {\hat{s}}^{′} ) ,\end{matrix} \qquad ( 5 )$$

with δ\tilde{μ}ₛ(r,ŝ·ŝ′)=δμₛ(r) ƒ(ŝ·ŝ′)+μₛ[ƒᵣ(ŝ·ŝ′)−ƒ(ŝ·ŝ′)].

In Eq. 5, μ_{α} and μₛ are the average absorption and scattering coefficients, respectively: δμ_{α}(r)(<<μₐ) and δMₛ(r)(<<μₛ) are the perturbations caused by the tumors.

Generally, the local variation of the phase function ƒᵣ(ŝ·ŝ′) from the global phase function ƒ(ŝ·{circumflex over (s+EE′) may not be small for some angles, but Eq. (4) requires that such variations cancel each other after integrating over all solid angles. Thus treat δ\tilde{μ})}_{S}(r,ŝ·ŝ′) as a small perturbation.

Under variations given by Eq. (5), Eq. (3) can be solved using perturbation theory. For the case of a point source,

$$Q ( r , \hat{s} , t ) = \frac{1}{4 π} δ ( r - {r}_{0} ) δ ( t - t ) , \qquad ( 6 )$$

Eq. (3) is the equation for the Green's function G (r,t|r₀t₀), which has an expansion

G_{ŝ}(r,t|r₀,t₀)=G_{ŝ}⁽⁰⁾(r,t⁰|r₀,t₀)+G_{ŝ}⁽¹⁾(r,t⁰|r₀,t₀)+. . . (7)

with G_{ŝ}⁽⁰⁾(r,t|r₀,t₀) being the normal Green's function in the absence of perturbation and G_{ŝ}⁽¹⁾(r,t|r₀,t₀) the first order correction due to the perturbation. Substituting Eqs. (5)-(7) into Eq. (3) and keeping terms only up to the first order, we have:

$$\frac{1}{c} \frac{\partial {G}_{\hat{s}}^{( 0 )} ( r , t \vert {r}_{0} {t}_{0} )}{\partial t} + \hat{s} \cdot ∇ {G}_{\hat{s}}^{( 0 )} ( r , t \vert {r}_{0} {t}_{0} ) + ( {μ}_{a} + {μ}_{s} ) {G}_{s}^{( 0 )} ( r , t \vert {r}_{0} {t}_{0} ) - {μ}_{s} {\int }_{4} {\int }_{π} {G}_{{\hat{s}}^{′}}^{( 0 )} ( r , t \vert {r}_{0} , {t}_{0} ) f ( \hat{s} \cdot {\hat{s}}^{′} ) ⅆ {Ω}^{′} = \frac{1}{4 π} δ ( r - {r}_{0} ) δ ( t - {t}_{0} ) \qquad ( 8 )$$

and

$$\frac{1}{c} \frac{\partial {G}_{\hat{s}}^{( 0 )} ( r , t \vert {r}_{0} {t}_{0} )}{\partial t} + \hat{s} \cdot ∇ {G}_{\hat{s}}^{( 1 )} ( r , t \vert {r}_{0} {t}_{0} ) + ( {μ}_{a} + {μ}_{s} ) {G}_{s}^{( 1 )} ( r , t \vert {r}_{0} {t}_{0} ) - {μ}_{s} {\int }_{4} {\int }_{π} {G}_{{\hat{s}}^{′}}^{( 1 )} ( r , t \vert {r}_{0} , {t}_{0} ) f ( \hat{s} \cdot {\hat{s}}^{′} ) ⅆ {Ω}^{′} = - [ δμ ( a ) ( r ) + {δμ}_{s} ( r ) ] {G}_{\hat{s}}^{( 0 )} ( r , t \vert {r}_{0} , {t}_{0} ) + {\int }_{4} {\int }_{π} {G}_{{\hat{s}}^{′}}^{( 0 )} ( r , t \vert {r}_{0} , {t}_{0} ) δ {\tilde{μ}}_{s} ( r , \hat{s} \cdot \hat{s} ) ⅆ {Ω}^{′} . \qquad ( 9 )$$

Equation (9) can be solved through the adjoint equation of Eq. (8), which is:

$$- \frac{1}{c} \frac{\partial {G}_{\hat{s}}^{( 0 )} ( r , t \vert {r}_{0} {t}_{0} )}{\partial {t}_{0}} + \hat{s} \cdot {∇}_{0} {G}_{\hat{s}}^{( 0 )} ( r , t \vert {r}_{0} {t}_{0} ) + ( {μ}_{a} + {μ}_{s} ) {G}_{s}^{( 0 )} ( r , t \vert {r}_{0} {t}_{0} ) - {μ}_{s} {\int }_{4} {\int }_{π} {G}_{{\hat{s}}^{′}}^{( 0 )} ( r , t \vert {r}_{0} , {t}_{0} ) f ( \hat{s} \cdot {\hat{s}}^{′} ) ⅆ {Ω}^{′} = \frac{1}{4 π} δ ( r - {r}_{0} ) δ ( t - {t}_{0} ) . \qquad ( 10 )$$

It can be shown that the solution to Eq. (9) satisfies:

$$\frac{1}{4 π} {\int }_{4} {\int }_{π} ⅆ Ω {G}_{\hat{s}}^{( 1 )} ( r , t \vert {r}_{0} , {t}_{0} ) = - {\int }_{{t}_{0}}^{{t}_{′}^{+}} ⅆ {t}^{′} \int {ⅆ}^{3} {r}^{′} [ {δμ}_{a} ( {r}^{′} ) + {δμ}_{s} ( {r}^{′} ) ] {\int }_{4} {\int }_{π} ⅆ {G}_{\hat{s}}^{( 0 )} ( r , t \vert {r}_{0} , {t}_{0} ) {G}_{\hat{s}}^{( 0 )} ( {r}^{′} , {t}^{′} \vert {r}_{0} , {t}_{0} ) + {\int }_{{t}_{0}}^{{t}_{′}^{+}} ⅆ {t}^{′} \int {ⅆ}^{3} {r}^{′} {\int }_{4} {\int }_{π} ⅆ {Ω}^{′} {\int }_{4} {\int }_{π} ⅆ Ω {G}_{{\hat{s}}^{′}}^{( 0 )} ( r , t \vert {r}_{0} , {t}_{0} ) {G}_{\hat{s}}^{( 0 )} ( {r}^{′} , {t}^{′} \vert {r}_{0} , {t}_{0} ) δ {\tilde{μ}}_{s} ( r , \hat{s} \cdot {\hat{s}}^{′} ) - {\int }_{{t}_{0}}^{{t}_{′}^{+}} ⅆ {t}^{′} {∯}_{\partial v} ⅆ {A}^{′} \cdot {\int }_{4} {\int }_{π} ⅆ Ω \hat{s} {G}_{\hat{s}}^{( 0 )} ( r , t \vert {r}^{′} , {t}^{′} ) {G}_{\hat{s}}^{( 1 )} ( {r}^{′} , {t}^{′} \vert {r}_{0} , {t}_{0} ) . \qquad ( 11 )$$

Under the assumption of uniqueness of the solution to the transport equation, Eq. (11) can be reduced to:

$$\frac{1}{4 π} {G}_{\hat{s}}^{( 1 )} ( r , t \vert {r}_{0} , {t}_{0} ) = - {\int }_{{t}_{0}}^{{t}^{+}} ⅆ {t}^{′} \int {ⅆ}^{3} {r}^{′} {δμ}_{a} ( {r}^{′} ) {G}_{\hat{s}}^{( 0 )} ( r , t \vert {r}^{′} , {t}^{′} ) {G}_{\hat{s}}^{( 0 )} ( {r}^{′} , {t}^{′} \vert {r}_{0} , {t}_{0} ) - {\int }_{{t}_{0}}^{{t}^{+}} ⅆ {t}^{′} \int {ⅆ}^{3} {r}^{′} {δμ}_{s} ( {r}^{′} ) {G}_{\hat{s}}^{( 0 )} ( r , t \vert {r}^{′} , {t}^{′} ) {G}_{\hat{s}}^{( 0 )} ( {r}^{′} , {t}^{′} \vert {r}_{0} , {t}_{0} ) + {\int }_{{t}_{0}}^{{t}^{+}} ⅆ {t}^{′} \int {ⅆ}^{3} {r}^{′} {\int }_{4} {\int }_{π} ⅆ {Ω}^{′} {G}_{\hat{s}}^{( 0 )} ( r , t \vert {r}^{′} , {t}^{′} ) {G}_{{\hat{s}}^{′}}^{( 0 )} ( {r}^{′} , {t}^{′} \vert {r}_{0} , {t}_{0} ) δ {\tilde{μ}}_{s} ( {r}^{′} , \hat{s} \cdot {\hat{s}}^{′} ) - {\int }_{{t}_{0}}^{{t}^{+}} ⅆ {t}^{′} {∯}_{\partial v} ⅆ {A}^{′} \cdot \hat{s} {G}_{\hat{s}}^{( 0 )} ( r , t \vert {r}^{′} , {t}^{′} ) {G}_{\hat{s}}^{( 1 )} ( {r}^{′} , {t}^{′} \vert {r}_{0} , {t}_{0} ) . \qquad ( 12 )$$

The first term on the right hand side of Eq. (12) is the correction due to absorption variations, the second and the third terms contain the correction due to scattering variations; the third term also includes an extra correction due to phase function variations. The last term in Eq. (12) is the surface integral and is related to the boundary condition. For boundary conditions commonly used, such as the zero-boundary condition in our system described below, the surface integral contributes at higher order and can be discarded. In order to simplify the formulation, define the point spread function as:

PSF(r,t;r′;r₀,t₀)=4π∫_{-∞}^{t+}dt′G_{ŝ}⁽⁰⁾(r,t|r′,t′)G_{ŝ}⁽⁰⁾(r′,t′|r₀, t₀) (13)

Therefore, Eq. (7) and Eq. (12) can be rewritten as:

−[G_{ŝ}(r,t|r₀,t₀)−G_{ŝ}⁽⁰⁾(r,t|r₀,t₀)]=∫d³r′δμ_{α}(r′)·PSF(r,t;r′;r₀t₀) (14)

Note the remarkable similarity between Eq. (2) and Eg/ (14). Physically,

Note the remarkable similarity between Eq. (2) and Eq. (14). Physically, PSF(r,t;r′;r₀,t₀) is the probability that a photon is injected at the source point r₀ at time t₀ passes through the field point r′ before time t, and is collected by the detector at point r at time t. It represents the photon path distribution at time t between the light source and the detector. In the ballistic limit, the photon path distribution in the transverse direction (i.e., direction perpendicular to r−r₀) shrinks to a δ-function:

$$P S F ( r , t ; {r}^{′} ; {r}_{0} , 0 ) {\to }^{ t \to \vert r - {r}_{0} \vert / c } {δ}^{⊥} ( r - {r}_{0} ) . \qquad ( 15 )$$

Thus, the volume integral on the r.h.s. of Eq. (14) reduces to a line integral along r−r₀, and Eq. (2) and Eq. (14) are essentially identical.

It is important to note that the derivation of Eq. (14) does not employ the assumptions of the diffusion approximation. The Green's function G_{ŝ}⁽⁰⁾(r,t|r′,t′) in Eq. (13) can be calculated using various models. Three examples are the path integral solutions, the random walk solution, and the conventional diffusion approximation solution. Further details regarding time gated imaging methods can be found in U.S. Pat. No. 5,919,140, the entire contents of which is incorporated herein by reference. In fact, these reconstructions show that the conventional diffusion solution is inadequate for imaging with early arriving photons. It predicts a point spread function which is too wide and renders images which are too small. A solution satisfying causality is required.

For the purpose of comparison, consider the diffusion approximation:

$$\{ \begin{matrix}{G}_{s}^{( 0 )} ( r , t \vert {r}_{0} , {t}_{0} ) ≅ \frac{1}{4 π} {G}^{( 0 )} ( r , t \vert {r}_{0} , {t}_{0} ) - \frac{1}{4 π} \frac{1}{{μ}_{t r}} \hat{s} \cdot ∇ {G}^{( 0 )} ( r , t \vert {r}_{0} , {t}_{0} ) \\ {G}_{s}^{( 1 )} ( r , t \vert {r}_{0} , {t}_{0} ) ≅ \frac{1}{4 π} {G}^{( 1 )} ( r , t \vert {r}_{0} , {t}_{0} ) - \frac{1}{4 π} \frac{1}{{μ}_{t r}} \hat{s} \cdot ∇ {G}^{( 1 )} ( r , t \vert {r}_{0} , {t}_{0} )\end{matrix} \qquad ( 16 )$$

where μₜᵣ is the transport scattering property defined as μₜᵣ=μ_{α}+μ′ₛ, and μ′ₛ=(1−g)μₛ with g the mean cosine of the forward scattering angle. It can be shown from Eq. (11) that the first order correction to the Green's function is:

$${G}^{( 1 )} ( r , t \vert {r}_{0} , {t}_{0} ) = - \int {ⅆ}^{3} {r}^{′} {δμ}_{a} ( {r}^{′} ) {\int }_{{t}_{0}}^{{t}^{+}} ⅆ {t}^{′} [ {G}^{( 0 )} ( r , t \vert {r}^{′} , {t}^{′} ) {G}^{( 0 )} ( {r}^{′} , {t}^{′} \vert {r}_{0} , {t}_{0} ) + \frac{1}{3 {μ}_{t r}^{2}} ∇ {G}^{( 0 )} ( r , t \vert {r}^{′} , {t}^{′} ) \cdot {∇}^{′} {G}^{( 0 )} ( {r}^{′} , {t}^{′} \vert {r}_{0} , {t}_{0} ) ] - \frac{1}{3 {μ}_{t r}^{2}} \int {ⅆ}^{3} {r}^{′} {δμ}_{s}^{′} ( {r}^{′} ) {\int }_{{t}_{0}}^{{t}^{+}} ⅆ {t}^{′} ∇ {G}^{( 0 )} ( r , t \vert {r}^{′} , {t}^{′} ) \cdot {∇}^{′} {G}^{( 0 )} ( {r}^{′} , {t}^{′} \vert {r}_{0} , {t}_{0} ) . \qquad ( 17 )$$

The above equation is identical to the correction calculated directly from the diffuision equation with perturbation theory.

The present invention involves the series expansion method and the algebraic reconstruction technique (ART) for optical CT. The volume to be imaged is split into N×N×N cubic voxels. Each voxel is assigned a value representing the local average of the absorption distribution. The imaging problem is 2D in the case of X-ray. The linear attenuation in Eq. (2) is then simplified to a summation of the voxel values along the source-detector line. The ray sum can be exactly expressed as:

$${y}_{j} = {\sum }_{i = 1}^{{N}^{2}} {b}_{ij} {w}_{ij} {μ}_{i} . \qquad ( 18 )$$

In Eq. (18), yⱼ is the data of the jth measurement: w is a geometric factor related to the oblique angle of the jth measurement, and it equals the segment length of the jth ray within voxel i; and μᵢ is the local average of the absorption within voxel i. The summation on the right hand side of Eq. (18) is that of the N² voxels on the detection plane. In the case of X-rays, the factor bᵢⱼ is given as:

$${b}_{ij} = \{ \begin{matrix}1 , & the j th ray crosses pixel i ; \\ 0 , & other .\end{matrix} \qquad ( 19 )$$

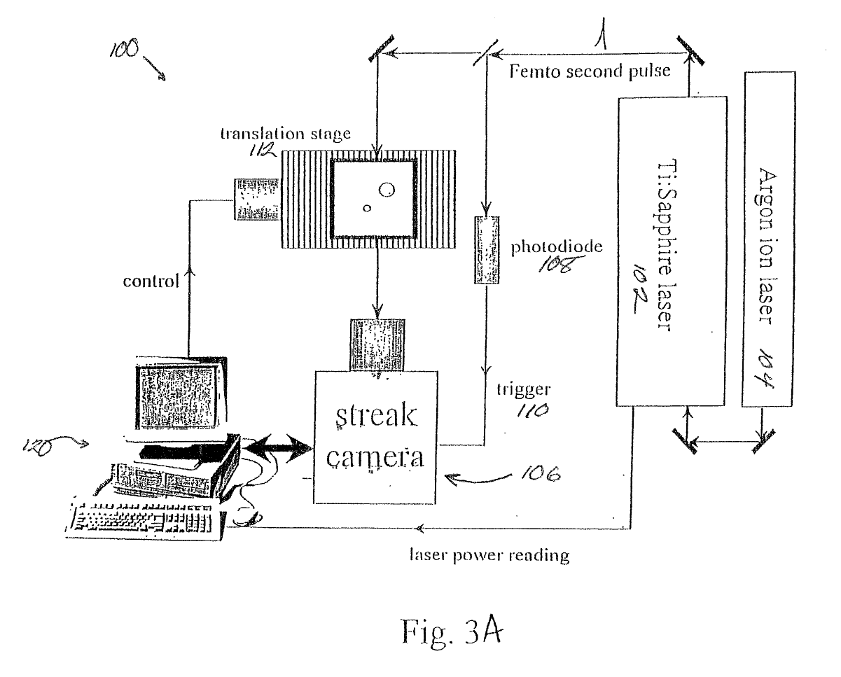

As illustrated in FIG. 2A, through the introduction of the weighting factor bᵢⱼ, the plane summation in Eq. (18) is actually a line summation. Those voxels which fall on the line give 100% contribution to the X-ray function, while those which fall away from the line give 0% contribution.

Our extension to optical CT is through the weighting factor bᵢⱼ. Because of scattering photons have different probabilities to follow different paths. Therefore, the contribution of each voxel to a source-detector measurement is weighted by the density of photon paths across it. The value of b for the optical case is the value of the photon path probability (FIG. 2B): Eq. (18) can then be directly applied. Recalling that the photon path is a 3D tube, summation over three dimensions is required. The generalized equation is:

$${y}_{j} = {\sum }_{i = 1}^{{N}^{3}} {p}_{ij} {w}_{ij} {⟨ {δμ}_{a} ⟩}_{i} . \qquad ( 20 )$$

This is exactly the discrete form of Eq. (14) at a fixed time t, if we set (21)

$$\{ \begin{matrix}{y}_{j} & = & - [ {G}_{s} ( {r}_{j} , t \vert {r}_{0} , {t}_{0} ) - {G}_{\hat{s}}^{( 0 )} ( {r}_{j} , t \vert {r}_{0} , {t}_{0} ) ] , \\ {⟨ {δμ}_{a} ⟩}_{i} & = & {δμ}_{a} ( {r}_{i} ) , \\ {p}_{ij} {w}_{ij} & = & P S F ( {r}_{j} , t ; {r}_{i} ; {r}_{0} r , 0 ) \cdot Δ v ,\end{matrix}$$

with Δv the volume element of each voxel. Equation (20) can be rewritten in a compact matrix form

y=Rx+n (22)

where y is the measurement vector (dimension M), x is the image vector (dimension N³), R is the projection matrix containing geometric and photon path information (dimension M×N³), and n denotes the error vector.

In image reconstruction, apply the additive ART of X-ray CT directly to optical CT. Since the imaging problem can be either overdetermined or underdetermined, it is inappropriate to solve the equation y=Rx directly, as it may not have any solution at all, or it may have many solutions but none is appropriate. The estimation of the image vector is usually performed using optimization criteria, based on the error between forward model predictions and experimental data, as well as a priori information about the imaging region. One example is the so-called regularized least-squares criterion, which is a special case of the Bayesian estimate. The reconstruction of the sought-after image is performed through minimizing the function:

r²∥y−Rx[²+∥x−δμ₀∥² (23)

In Eq. (23), r is a parameter often called the signal-to-noise ratio in the literature δμ₀ is the pre-knowledge for the image vector (N³×1) and also used as the initial estimate of the image, and ∥. . . ∥² denotes the module square for the vector. Keeping the second term small ensures that the picture is not too far from the pre-knowledge, and keeping the first term small ensures that the picture is consistent with the measurements.

One iterative procedure to minimize Eq. (20) is to introduce two sequences of vectors, x^{(k)} and u^{(k)}, of dimensions N³ and M, respectively. Initially, x⁽⁰⁾ is set equal to δμ₀ and u⁽⁰⁾ to a zero vector. The iterative step (G. T. Herman, Image Reconstruction From Projections: The Fundamentals of Computerized Tomography (Academic Press, New York, N.Y., 1980)) which is incorporated herein by reference in its entirety is given by:

$$\{ \begin{matrix}{x}^{( k + 1 )} = {x}^{( k )} + r {c}^{( k )} {R}_{{i}_{k}} \\ {u}^{( k + 1 )} = {u}^{( k )} + {c}^{( k )} {e}_{{i}_{k}}\end{matrix} , \qquad ( 24 )$$

where

$${c}^{( k )} = λ r ( {y}_{{i}_{k}} - {\sum }_{j = 1}^{{N}^{3}} {R}_{{i}_{k} , j} {x}_{j}^{( k )} ) - \frac{{u}_{{i}_{k}}^{( k )}}{1 + {r}^{2} {‖ {R}_{{i}_{k}} ‖}^{2}} \qquad ( 25 )$$

with iₖ=k(mod N³)+1. Rᵢ the transpose of the i-th row of the matrix R.e, the M dimensional column vector with 1 in the i-th row and 0 elsewhere, and λ is a real number called relaxation parameter. It has been proven that as long as 0<λ<2 the sequence {x^{(k)}} converges in the limit to the minimizer of Eq. (23).

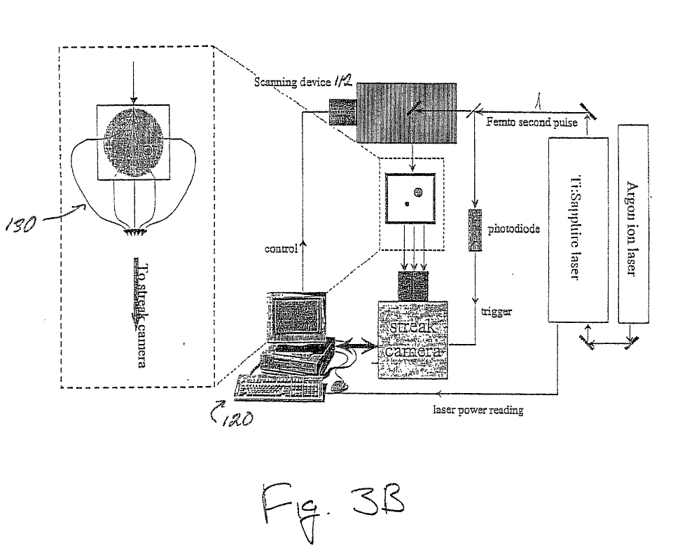

In order to demonstrate this optical CT technique, measurements were made in a cubic glass container 6.35 cm on a side, filled with a tissue-like scattering medium. The cubic geometry was selected to simplify the theoretical modeling because of its regular boundaries. The scattering medium was a stock solution prepared with 1.072 μm diameter polystyrene beads (PolyScience, Inc.) at 0.27% concentration. The scattering and adsorption properties of the medium were determined in real time by fitting the transmitted diffuse light. A schematic diagram of the system 100 is presented in FIGS. 3A and 3B. The light source 102 was a Coherent Mira 900 mode-locked Ti:sapphire laser operated in femto-mode and pumped by a Coherent Inova 400 multiline argon ion laser 104. The wavelength was 800 nm, and the repetition rate was 76 MHZ. The pulse width was ˜150 fs. The detection system 106 was a Hamamatsu streak camera C5680 with M5675 Synchroscan Unit. A small portion of the laser beam was deflected by a quartz plate to a fast photodiode 108 (Hamamatsu C1808-02), which generates the triggering signal 110 for the streak camera. The cubic glass container was mounted on a translation stage 112 used to actuate relative movement between the source, the detector and the object to be scanned. A Pentium-II computer 120 served as a central controlling and data acquisition unit. In order to monitor the laser power drift during the scan, a DT2801-A card (Data Translation, Inc.) was installed on the computer and programmed to record the laser power voltage from the control box of the light source, and it also served to drive the stepper motor of the translation stage. Total automation of data acquisition for a full line scan was achieved through programming the user interface of the streak camera software. The streak camera was operated in analog mode. It converted the temporal evolution of light signals into vertical streak images. The transmitted light was collected with four coherent fiber bundles 130. Each fiber bundle had a 500 μm core diameter and consisted of ten thousand single mode silica fibers. The proximal ends of the four fibers were bundled together to increase the collection area. The overall time resolution of the detection system was set to 30 ps.

The container was placed in the sample holder on the translation stage. Multiple objects were embedded inside the turbid medium at fixed positions. The sample holder was designed so that the top, bottom, left and right boundaries of the container were totally black. The laser pulses were delivered into the medium at the front side, and the transmitted signals were collected at the opposite side. The incoming laser beam and the collection fibers were aligned in a coaxial geometry. Surface scans were conducted on the XZ and YZ directions, so that a 3D image of the absorption distribution could be reconstructed. The scan area was a 4 cm×4 cm square along each direction. The scans were 2 mm per step in the horizontal and vertical directions, resulting in 2 projections, 882 total scan measurements. The data acquisition time for each point was 8s. Either one or two opaque objects were embedded in the medium. In each case, the scans were first carried out for the XZ plane, and then the container was rotated by 90° and the scans continued for the YZ plane.

The calculation of the point spread function requires knowledge about the average scattering and absorption properties of the scattering medium (μ′ₛ and μ_{α}) To determine μ′ₛ and μ_{α}, the coaxial transmission signal of a 2.2 ns time window was collected, with the absorbers removed from the scattering medium and the source-detector aligned at the center of the X-surface. This time-dependent curve was fitted using the diffusion approximation solution with zero-boundary condition. The best fit was given by μ′ₛ=7.38 cm⁻¹ and μ_{α}=0.03 cm⁻¹. These values are similar to those of human breast tissue.

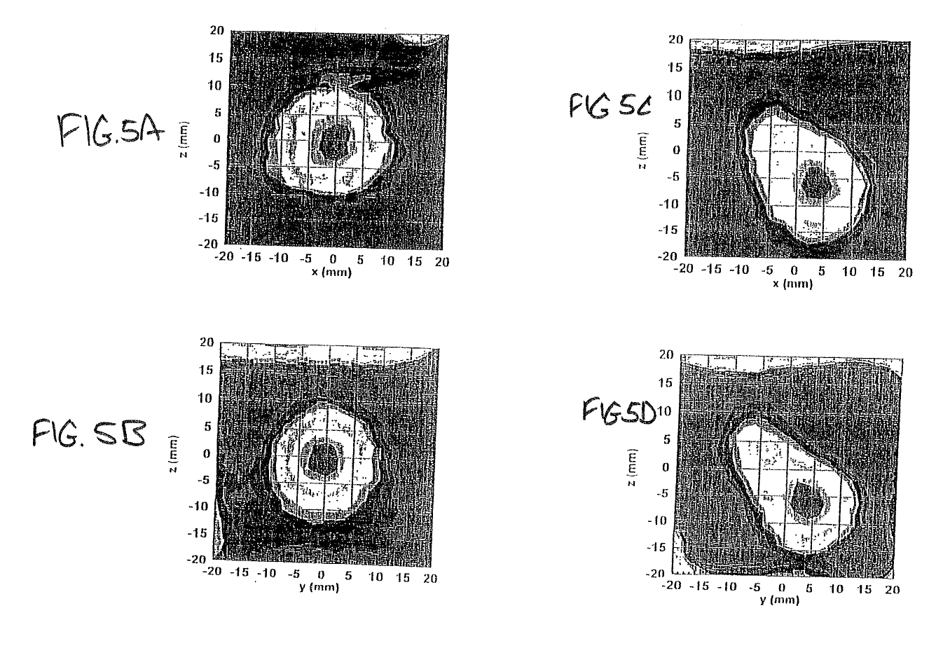

In these measurements, data was collected for two configurations. In the first case, an 8 mm diameter black sphere was placed at the center of the container (“one object configuration”). In the second case, a black sphere 8 mm in diameter was placed at (0.56, 56.−0.56) cm from the center, and a second black sphere 6 mm in diameter was placed at (−0.56.−0.56.56) cm from the center (“two object configuration”). For each case, scanning was performed on the surfaces in 2 mm steps, and 882 time evolution curves of the transmission signals were measured, one for each scanning point. Because the source-detector distance remained the same for all the 882 measurements, the time scale was the same and the intensities can be compared at the same time point. When the source-detector line crosses the absorber there is a drop in signal level, and vice versa. The projection intensity diagrams can be easily created by plotting the contour map of the intensities of all the 882 time evolution curves at the same time point. To achieve higher counts and reduce noise, such intensities can be summations of counts over a time window comparable to the time resolution of the detecting system. The selection of the time point is based on the trade-off of spatial resolution and the absorbers have weaker intensity. The absorbers appear as shadows on the projection intensity diagrams. The projections of one embedded absorber (FIGS. 5A and 5B) are different from those of two embedded absorbers (FIGS. 5C and 5D). FIGS. 5A-5D are referred to as the zero-th order images. They already show features such as the central positions of the embedded objects, though the images are scrambled due to scattering.

Before processing the data of FIGS. 5A-5D, artifacts at the edges due to tiny air bubbles and asymmetry of the surface were removed manually, and a grid was created. In this analysis choose the 2-mm scanning step as the size of each grid, resulting in 21³=9261 voxels. The inverse is underdetermined, as 9261 voxel values are reconstructed from 882 measurements.

The image reconstruction procedure was then applied in two steps. First, the straight line PSF of X-ray CT was applied to the data using ART (Eqs. (24) and (25)) with an initial estimate of δμ=0 (See Eq. (23)) and “signal-to-noise” r=60. Second, the resulting image, obtained with the straight line PSF, was then used as an initial estimate and applied to the data using a particular photon migration PSF. The “signal-to-noise ratio” in Eq. (23) was again set to r=60, and the relaxation parameter in Eq. (25) was set to λ=1. The reconstructions were computed with point spread functions calculated from both the diffusion approximation and the causality corrected solutions (see below). The number of iteration steps was set to 2×10⁵. On a Pentium-II 450 MHz PC, each reconstruction took ˜40 seconds. The number of iteration steps was increased to 2×10⁶ without observing much improvement.

The second step involves the calculation of the PSF from solutions to the transport equation. The diffusion approximation solution is widely used in the literature. However, it is well known that this solution violates causality and breaks down in the early time regime. Solutions incorporating causality have been worked out for models based on random walk and path integral theories. A Green's function which incorporates causality and is valid for early arriving photons has been used. This Green's function is constructed from the diffusion approximation Green's function G(r,t|r₀,t₀) by replacing the time t₀ at which the pulse is injected into the medium with t₀+tᵢᵣ with tᵢᵣ the time of flight for unscattered photons to propagate from the source to the detector. Physically, this procedure takes into account the fact that diffusion starts only after the light pulse traverses the medium.

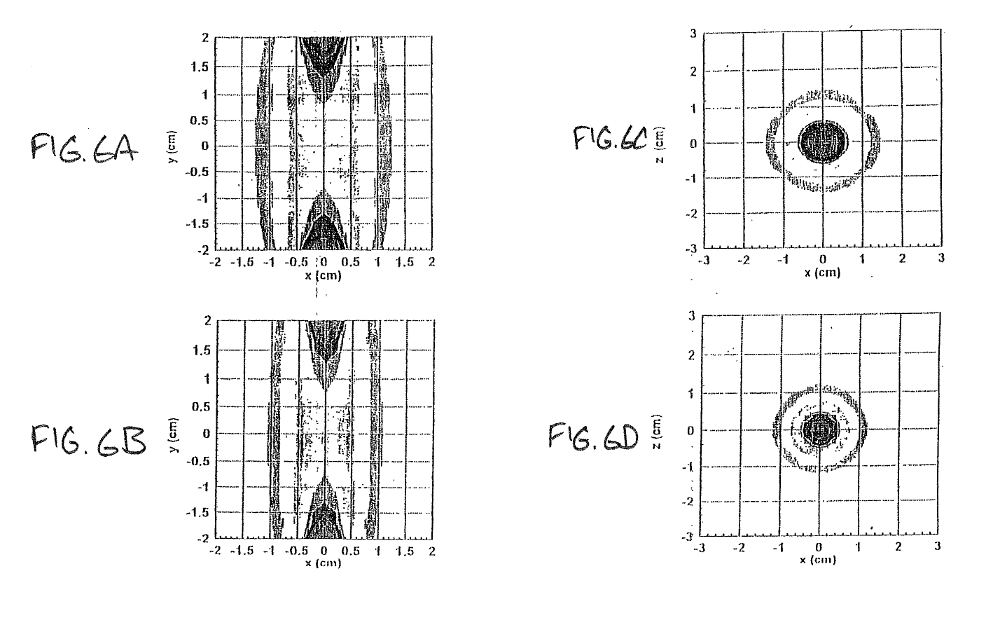

FIGS. 6A-6D show the point spread functions calculated with the diffusion approximation solution and with the causality corrected solution. Note that all contour maps in FIGS. 6A-6D correspond to the time window of the experimental data for the two-object case. The point spread function calculated using the diffusion approximation is wider than that using the causality modified procedure.

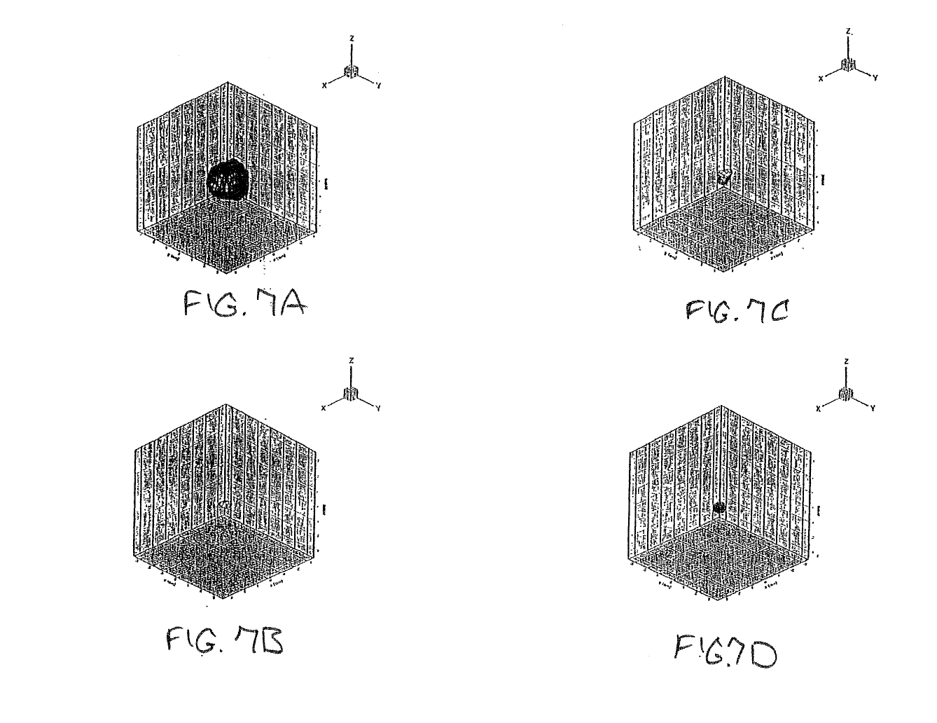

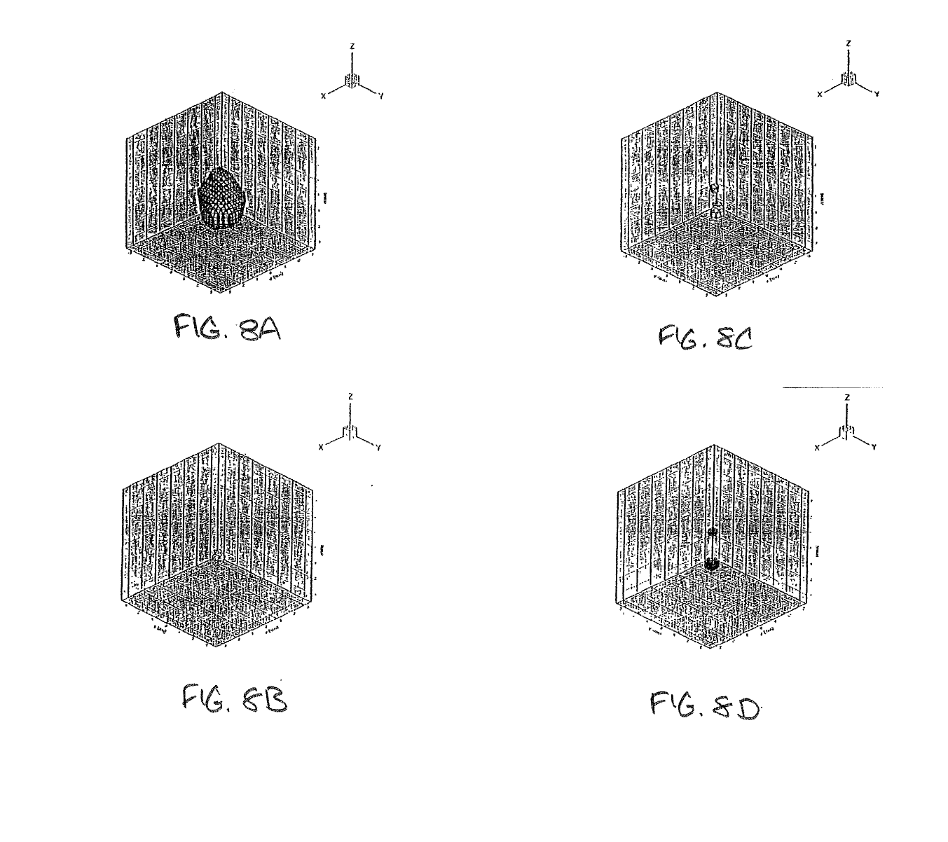

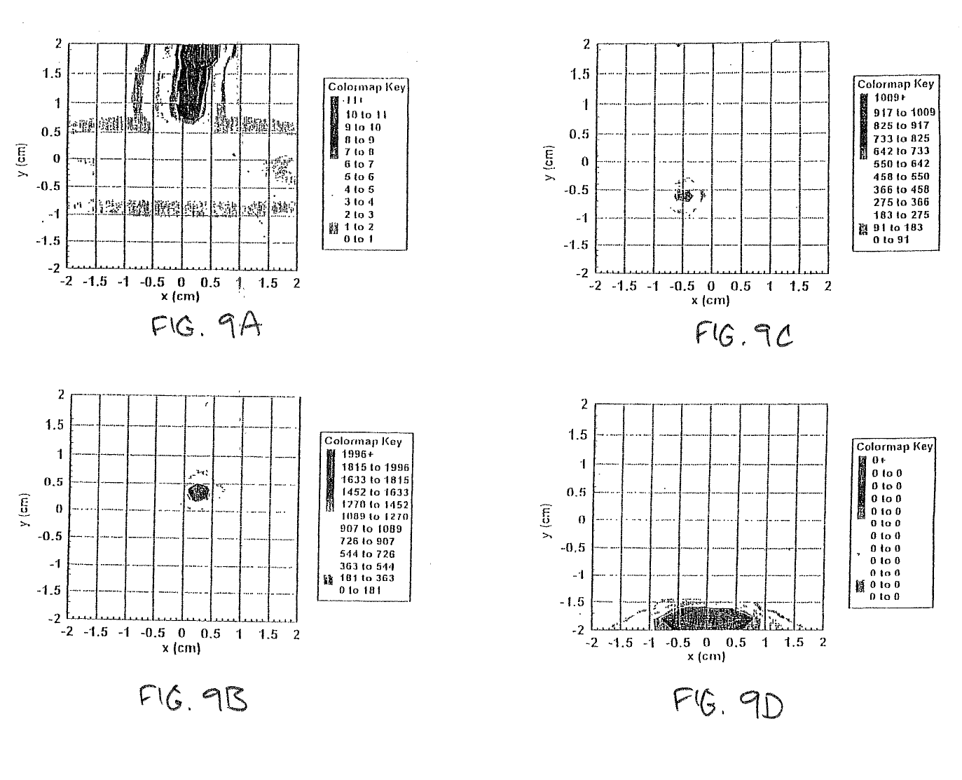

The image reconstruction results for X-ray (straight line PSF), diffusion approximation, causality correction, and the actual configuration are presented in the contour plots of FIGS. 7A-7D and FIGS. 8A-8D. FIGS. 9A-9D present characteristic slices of FIG. 8C in 2D contour maps. The slices are picked at planes z=−10 mm z=−6 mm z=2 mm and z=10 mm. As clearly seen in FIGS. 7A-7D and 8A-8D, the images reconstructed with the straight line path (X-ray CT) do not have high resolution, while the images reconstructed with the diffusion approximation are too small compared with the actual size of the spheres, especially for the two object case, for which the smaller object is invisible. Due to the noise in the experimental data, the reconstructed images for the one object configuration exhibit some distortions. Remarkably, for the two object configuration the image reconstructed with the causality correction give correct sizes of the embedded objects. As shown in FIG. 9, the voxel values off the objects are nearly zero which naturally results from the inverse procedure. In FIG. 8C, the size and the position of the 8-mm object and the size of the 6-mm object are correct, while the position of the 6-mm object is off from its actual position by 2 mm. This may indicate that a cross-talk exists in the inverse when two objects are embedded.

The point spread function calculated with the conventional diffusion approximation solution is too wide for the early time window of our data. The reconstructed images in this case are too small compared with the actual size of the objects. Especially for the two object configuration, the smaller absorber is basically invisible in the reconstructed image. On the other hand, the same calculations done with the causality correction provide improved resolution.

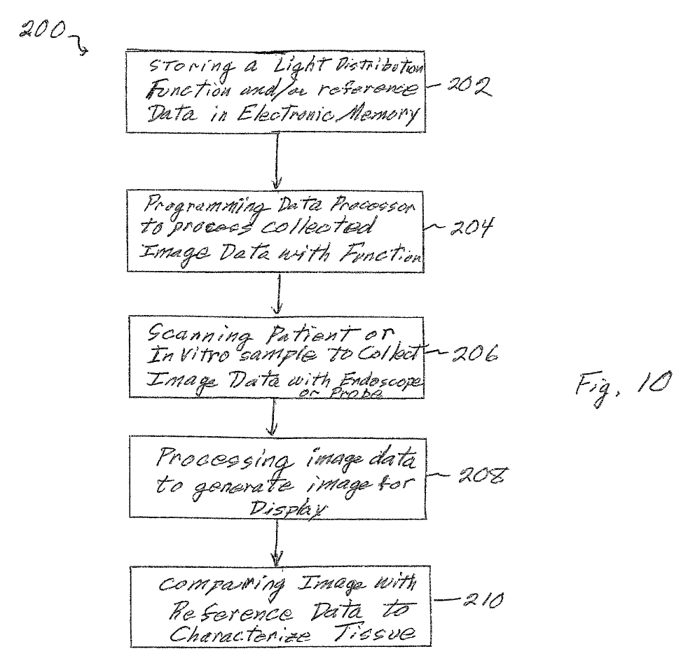

FIG. 10 illustrates a process sequence 200 in accordance with a preferred embodiment of the invention. After storing 202 a light distribution function and/or any reference data in electronic memory and programming 204 the computer to process the collected image data for a particular type of anatomy or tissue structure such as a concerous lesion, the patient or biopsied sample is scanned 206 with an endoscope or probe. The collected data is processed 208 to generate an image for display and for further processing 210.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of imaging an object comprising:
providing a light distribution function, the function having a scattering component and an absorption component;
directing light onto an object to be imaged;
detecting light emitted by the object;
forming an electronic representation of the object with the detected light; and
processing the electronic representation using the light distribution function to form an image of the object.

2. The method of claim 1 further comprising directing light onto the object, the light having a wavelength in the range of 700 nm to 900 nm.

3. The method of claim 1 wherein the object comprises tissue such that the method further comprises forming an image of the tissue.

4. The method of claim 1 further comprising providing a light source, a detector, and a data processor connected to the detector.

5. The method of claim 1 further comprising providing the light distribution function including a series expansion.

6. The method of claim 1 further comprising providing a collection time during which light is detected, the collection time being less than 1000 ps.

7. The method of claim 4 wherein the step of providing a light source comprises providing a laser.

8. The method of claim 4 wherein the step of providing detector comprises providing a streak camera.

9. The method of claim 1 wherein the light distribution function comprises a point spread function.

10. The method of claim 9 further comprising providing a plurality of weighting functions.

11. The method of claim 1 further comprising determining a size of a cancerous lesion in tissue.

12. A system for imaging an object comprising:
a data processor having a light distribution function, the function having a scattering component and an absorption component;
a light delivery system that delivers light onto an object to be imaged;
a light sensor that detects light emitted by the object, the sensor being connected to the data processor such that an electronic representation of the object is formed with the detected light, the electronic representation being processed using the light distribution function to form an image of the object.

13. The system of claim 12 further comprising directing light onto the object, the light having a wavelength in the range of 700 nm to 900 nm.

14. The system of claim 12 wherein the object comprises tissue and further comprising a display connected to the data processor that displays an image of the tissue.

15. The system of claim 12 further comprising a light source aligned with the detector.

16. The system of claim 12 further comprising a light distribution function including a series expansion.

17. The system of claim 12 further comprising a controller that controls a collection time during which light is detected, the collection time being less than 1000 ps.

18. The system of claim 15 wherein the light source comprises a laser.

19. The system of claim 12 wherein the sensor comprises a streak camera.

20. The system of claim 12 further comprising a scanner that provides relative movement between the object being imaged and the sensor.

21. The system of claim 20 further comprising a controller that controls, the scanner, a gated detector, the light source and data processing.

22. The system of claim 12 further comprising a plurality of light distribution function.

23. The system of claim 12 further comprising a fiber optical light coupler.

24. The system of claim 12 further comprising a probe for insertion into the body to deliver light to tissue.

25. The system of claim 12 further comprising a plurality of weighting functions.

26. A method of imaging a patient comprising:
providing a light distribution function, the function having a scattering component and an absorption component;
providing an electronic representation of tissue within the patient; and
processing the electronic representation using the light distribution function to form an image of the object.

27. The method of claim 26 wherein light collected from the patient has a wavelength in the range of 700 nm to 900 nm.

28. The method of claim 26 further comprises forming an image of a cancerous lesion within the tissue.

29. The method of claim 26 further comprising providing a data processor programmed with the light distribution function.

30. The method of claim 26 wherein the light distribution function including a series expansion.

31. The method of claim 26 further comprising providing a collection time during which light is collected from the patient the collection time being less than 1000 ps.

32. The method of claim 26 further comprising a detector such as a streak camera.

33. The method of claim 26 further comprises providing alight distribution function having a series expansion component.

34. The method of claim 26 wherein the light distribution function comprises a point spread function.

35. The method of claim 34 further comprising providing a plurality of weighting functions.

36. The method of claim 26 further comprising determining a size of a cancerous lesion in tissue.

37. The method of claim 26 further comprising collecting light with a fiber optic device.

38. The method of claim 26 further comprising defining an imaging volume having a plurality of voxels within the body being imaged, each voxel having a weighting factor.

39. The method of claim 26 wherein the light distribution function includes a transport equation approximation.

40. The method of claim 26 wherein the light distribution function defines a plurality of light paths having a cross-sectional area, the area being less than diffusion approximation of the area.
